(19) United States
(12) Patent Application Publication
(10) Pub. No.: US 2025/0205505 A1
(43) Pub. Date: Jun. 26, 2025

(54) DEFIBRILLATOR AND DEFIBRILLATION ENERGY CHARGING METHOD

(71) Applicant: FUKUDA DENSHI CO., LTD., Tokyo (JP)

(72) Inventors: Katsuya IDE, Tokyo (JP); Ryosuke SAKAI, Tokyo (JP); Taichi IWASAKI, Tokyo (JP); Koki OYA, Tokyo (JP); Takashi YAMAKI, Tokyo (JP); Kohei MITSUMARU, Tokyo (JP); Hiroshi HINO, Tokyo (JP); Yuzuru SAKAI, Tokyo (JP)

(21) Appl. No.: 18/989,006

(22) Filed: Dec. 20, 2024

(30) Foreign Application Priority Data
Dec. 26, 2023 (JP) 2023-219914

(51) Int. Cl. A61N 1/39; A61N 1/362

(52) U.S. Cl. CPC A61N 1/3981; A61N 1/3625; A61N 1/3904

(57) ABSTRACT

A defibrillator of the present disclosure includes: a battery; a capacitor that stores defibrillation energy; a controller that is configured to control charging of the capacitor by the battery; and a voltage analyzer that measures an output voltage of the battery when the capacitor is charged by the battery. The controller is configured to control the charging of the capacitor by the battery such that the charging of the capacitor by the battery is divided into at least pre-charging and additional charging subsequent to the pre-charging, and the controller is further configured to control, based on the output voltage of the battery measured by the voltage analyzer during the pre-charging, a charging rate during the additional charging.

### CROSS REFERENCE TO RELATED APPLICATIONS

This application is entitled to and claims the benefit of Japanese Patent Application No. 2023-219914, filed on Dec. 26, 2023, the disclosure of which including the specification, drawings and abstract is incorporated herein by reference in its entirety.

### TECHNICAL FIELD

The present disclosure relates to a defibrillator and a defibrillation energy charging method, in particular, relates to a defibrillator that charges a capacitor with defibrillation energy by using a battery.

### BACKGROUND ART

A defibrillator has been widely used as an apparatus for treating ventricular fibrillation (Vf). An automated external defibrillator (AED) is well known as a representative defibrillator. The defibrillator treats ventricular fibrillation or pulseless ventricular tachycardia (pulseless VT) of a patient who suffers from the ventricular fibrillation or pulseless ventricular tachycardia, by discharging a pulse of a high voltage into the heart of the patient.

Such a defibrillator includes a battery, a capacitor, and a power switch. Before the defibrillation is performed, the capacitor is charged by the battery. The capacitor is electrically connected to a discharger such as an electrode pad or a paddle via the power switch. When the power switch is turned on, the defibrillation energy stored in the capacitor is discharged through the discharger to the patient's heart.

Such a defibrillators is described in, for example, Japanese Patent Application Laid-Open No. 2016-187438.

In a defibrillator, a primary battery is used as a battery, and a large-capacity capacitor is used as a capacitor for storing defibrillation energy.

For this reason, especially when the capacitor is charged by the battery in a state where the battery is depleted (in other words, in a state where the remaining battery level is low), the output voltage of the battery drops significantly. In addition to the capacitor, electronic components such as a central processing unit (CPU) are also connected to the battery, and the battery also applies a voltage to these electronic components. Accordingly, when the output voltage of the battery decreases too much during charging of the capacitor, there is a possibility that the operations of the entire defibrillator may be stopped.

It is conceivable that one method for solving this topic is to estimate the remaining battery level, and to control the output voltage based on the estimation result or to inform the user that the remaining battery level is low and urge the user to replace the battery. In the related art, the battery voltage in the no-load state is measured, and the remaining battery level is estimated based on the battery voltage, but the estimated value of the remaining battery level based on the voltage in the no-load state does not coincide with the actual remaining battery level. This is because the battery used in the defibrillator is a primary battery. Thus, the estimation of the remaining battery level in the related art is insufficient as a solution.

Further, another method is conceivable, in which a current value from the battery when the capacitor is charged is limited, thereby suppressing a decrease in the output voltage of the battery. When the current value is restricted inadvertently, however, the time required for charging the capacitor with a desired amount of defibrillation energy becomes long, which is not preferable. In particular, it is necessary to promptly treat patients suffering from ventricular fibrillation and pulseless ventricular tachycardia, and thus, there is a desire to make the time of energy charging into the capacitor as short as possible.

The present disclosure has been made in view of the above points, and an object thereof is to provide a defibrillator and a defibrillation energy charging method each capable of appropriately charging a capacitor in accordance with a remaining battery level.

### SUMMARY

An aspect of a defibrillator of the present disclosure includes:
a battery;
a capacitor that stores defibrillation energy;
a controller that is configured to control charging of the capacitor by the battery; and
a voltage analyzer that measures an output voltage of the battery.

The controller is configured to control the charging of the capacitor by the battery such that the charging of the capacitor by the battery is divided into at least pre-charging and additional charging subsequent to the pre-charging, and the controller is further configured to control, based on the output voltage of the battery measured by the voltage analyzer during the pre-charging, a charging rate during the additional charging.

An aspect of a defibrillation energy charging method of the present disclosure is a defibrillation energy charging method in a defibrillator, and includes:
pre-charging a capacitor by a battery;
additionally charging the capacitor by the battery after the pre-charging;
measuring an output voltage of the battery during the pre-charging; and
controlling, based on the measured output voltage, a charging rate during the additionally charging of the capacitor.

### BRIEF DESCRIPTION OF THE DRAWINGS

FIG. 1 is a block diagram illustrating a main configuration of a defibrillator in an embodiment;

FIG. 2 is a flowchart for describing a defibrillation energy charging operation in the embodiment;

FIGS. 3A and 3B are diagram illustrating the appearance of voltages during two-stage charging, where FIG. 3A illustrates the appearance of the output voltage of a battery, and FIG. 3B illustrates the appearance of the voltage between both ends (charging voltage) of a capacitor;

FIG. 4 is a flowchart illustrating the defibrillation energy charging operation by the defibrillator in the embodiment in more detail;

FIG. 5 is a diagram illustrating an operation transition with reference value Vstdmin as a threshold; and

FIG. 6 is a diagram illustrating the order of magnitudes of a plurality of thresholds Vpremin, VpreL, VpreM, and VpreH and an operation transition of the present embodiment using the thresholds.

### DESCRIPTION OF EMBODIMENTS

First, a history leading to the present disclosure will be described before describing an embodiment.

The inventors of the present disclosure have noted that a method for charging a capacitor from a battery in a defibrillator is performed in two separate stages: pre-charging and additional charging.

The pre-charging is performed immediately after the defibrillator is activated, and the additional charging is performed after the connection with the patient is confirmed. By charging a certain amount of defibrillation energy in advance through the pre-charging, the time from when an electrical shock is determined to be necessary until the energy charging is completed, that is, the time until the electrical shock is executed can be shortened.

The inventors of the present disclosure have considered that the remaining battery level can be estimated accurately by measuring the output voltage of the battery during pre-charging, as compared to by measuring the voltage of the battery in the no-load state. In addition, the inventors of the present disclosure have considered that, in a case where the charging rate during the additional charging is controlled based on the output voltage of the battery measured during the pre-charging, the additional charging in which excessive voltage drop is suppressed can be performed according to the remaining battery level, which led to the present disclosure.

Hereinafter, an embodiment of the present disclosure will be described in detail with reference to the drawings.

### <1> Main Configuration of Defibrillator

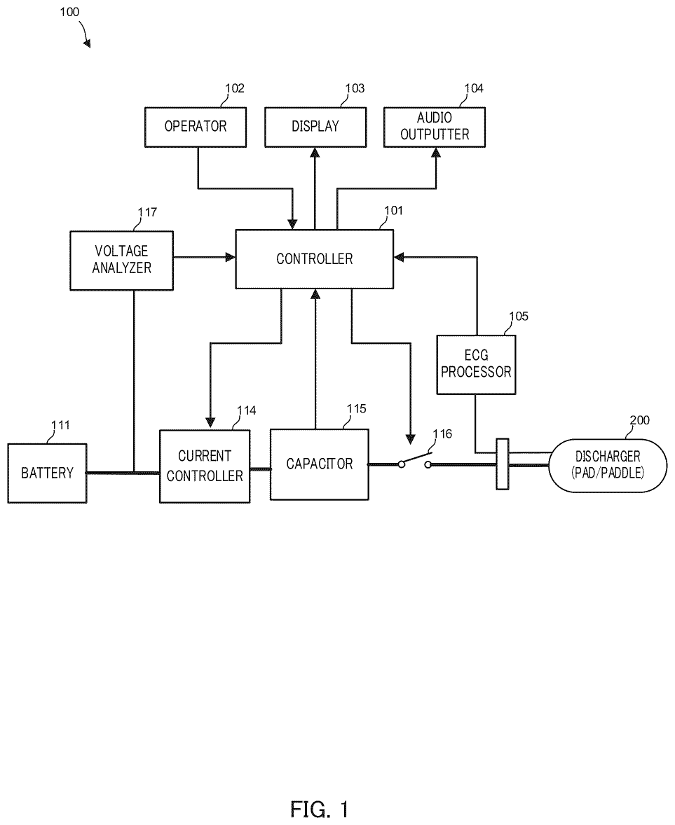

FIG. 1 is a block diagram illustrating a main configuration of defibrillator 100 in the present embodiment. Defibrillator 100 is called an external defibrillator or an AED. Discharger 200 that is an electrode pad or a paddle that comes into contact with a patient is connected to defibrillator 100.

Defibrillator 100 includes controller 101, operator 102, display 103, audio outputter 104, and electrocardiogram (ECG) processor 105. In addition, defibrillator 100 includes battery 111, current controller 114, capacitor 115, power switch 116, and voltage analyzer 117.

Controller 101 is implemented by a processor such as a central processing unit (CPU), and controls the operation of each section of defibrillator 100. Further, controller 101 implemented by the processor realizes, for example, a defibrillation energy charging method to be described later by executing a program.

Operator 102 includes, for example, a charge button, a shock button, and the like (not illustrated). Note that, in a case where discharger 200 is a paddle type, the shock button may be provided on the paddle. When the shock button is operated, the electric charge stored in capacitor 115 is output to discharger 200.

Further, defibrillator 100 is configured such that controller 101 can perform electrocardiogram analysis and also determine the necessity of defibrillation or the like based on the electrocardiogram acquired by ECG processor 105.

Battery 111 is used to charge capacitor 115 and generate defibrillation energy. Further, although not illustrated, battery 111 is also connected to electronic components such as controller (CPU) 101 in addition to capacitor 115, and battery 111 applies a voltage to these electronic components.

In the example of the present embodiment, battery 111 is a lithium primary battery. Further, battery 111 is a battery replaceably mounted in defibrillator 100 (that is, a replaceable battery), and is replaced by a new battery that has been fully charged, for example, every two years. Of course, the exchange timing of battery 111 is not limited to this.

Main battery 111 used as described above has an output voltage of, for example, 12 to 18 [V] in a state where no load (capacitor 115) is connected. In the example of the present embodiment, the output voltage of battery 111 is 15 [V] in a state where no load is connected.

Voltage analyzer 117 measures the output voltage of battery 111. Voltage analyzer 117 in the present embodiment measures the output voltage of battery 111 when capacitor 115 is charged by battery 111, and outputs the measurement result to controller 101. Note that, various voltage measurement circuits proposed heretofore can be applied as the configuration of voltage analyzer 117. For example, voltage analyzer 117 can be realized by a voltage measurement circuit including a monitoring IC (Integrated Circuit).

Controller 101 controls current controller 114 based on the voltage measured by voltage analyzer 117. Thus, the current value flowing from battery 111 to capacitor 115 is controlled based on the voltage measured by voltage analyzer 117. Current controller 114 is configured to include, for example, a switching element, and is capable of controlling the current value by switching operation based on the switching frequency from controller 101.

This current control will be described in detail later.

Further, controller 101 calculates the defibrillation energy stored in capacitor 115 based on the voltage between both ends of the high-voltage capacitor that constitutes capacitor 115. When the defibrillation energy is equal to or greater than a predetermined value and the shock button is operated, controller 101 turns on power switch 116 and causes the electric charge stored in capacitor 115 to be output to discharger 200.

### <2> Operation of Embodiment

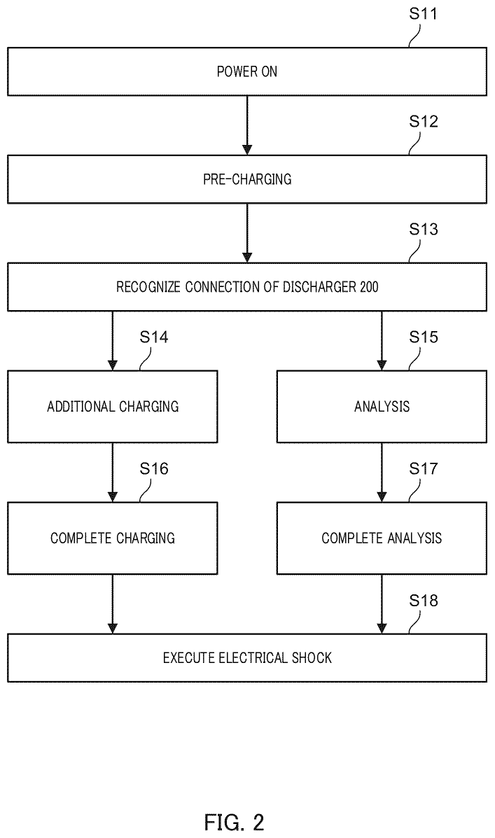

FIG. 2 is a flowchart for describing a defibrillation energy charging operation in defibrillator 100.

When defibrillator 100 is powered on in step S11, capacitor 115 is pre-charged by battery 111 in subsequent step S12. When defibrillator 100 recognizes the connection of discharger 200 in step S13 (that is, when controller 101 recognizes that discharger 200 is attached to the patient), capacitor 115 is additionally charged by battery 111 in step S14. Further, defibrillator 100 analyzes the patient's ECG or the like in step S15.

When defibrillator 100 completes the charging in step S16 and completes the analysis in step S17, defibrillator 100 executes an electrical shock by turning on power switch 116 in step S18.

Note that, in the example of FIG. 2, the additional charging (step S14) and the analysis (step S15) are performed in parallel, but, for example, the analysis may be performed first, and the additional charging may be performed when an analysis result indicating that an electrical shock is necessary is obtained.

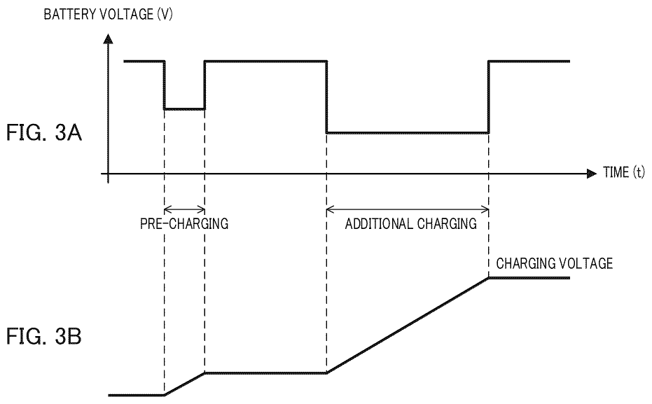

FIGS. 3A and 3B are diagrams illustrating the appearance of voltages in a case where energy is charged in two stages, where FIG. 3A illustrates the appearance of the output voltage of battery 111, and FIG. 3B illustrates the appearance of the voltage between both ends (charging voltage) of capacitor 115. Note here that, the charging rate (current flowing from battery 111 to capacitor 115) during the pre-charging is set to be equal to or less than the charging rate during the additional charging. Thus, a voltage drop of battery 111 during the pre-charging does not adversely affect the operations of the entire defibrillator.

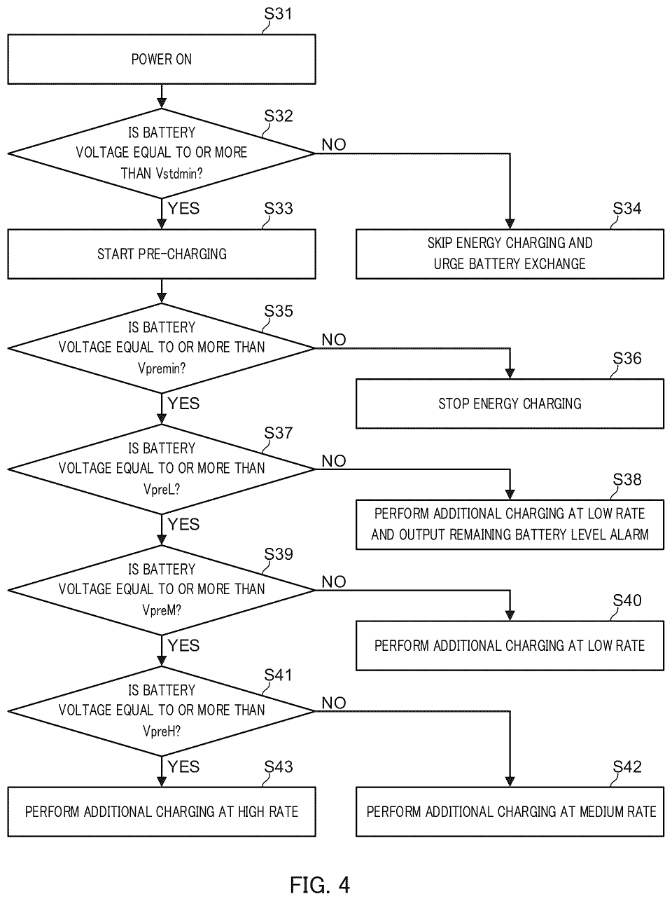

FIG. 4 is a flowchart illustrating the defibrillation energy charging operation by defibrillator 100 in the present embodiment in more detail.

When defibrillator 100 is powered on in step S31, controller 101 of defibrillator 100 determines in subsequent step S32 whether or not the output voltage (battery voltage) of battery 111 measured by voltage analyzer 117 is equal to or greater than reference voltage Vstdmin.

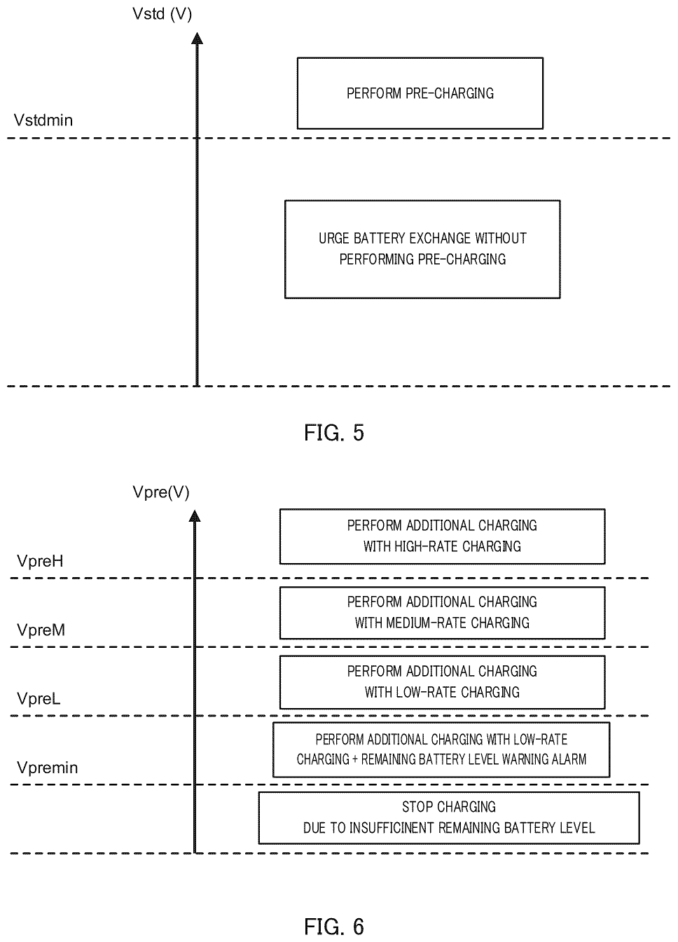

In a case where the battery voltage is less than reference value Vstdmin, controller 101 proceeds to step S34, skips the energy charging, and urges a battery exchange. Specifically, controller 101 causes current controller 114 to block the current, and causes display 103 and/or audio outputter 104 to output a display and/or sound for urging a battery exchange. In a case where the battery voltage is equal to or greater than reference value Vstdmin, on the other hand, controller 101 proceeds to step S33 and starts the pre-charging. Specifically, controller 101 controls current controller 114 such that a current corresponding to pre-charging flows in current controller 114. FIG. 5 is a diagram illustrating an operation transition with reference value Vstdmin as a threshold.

Subsequently, controller 101 selects the charging rate while determining stepwise whether the battery voltage is equal to or greater than a threshold. FIG. 6 is a diagram illustrating the order of magnitudes of a plurality of thresholds Vpremin, VpreL, VpreM, and VpreH and an operation transition of the present embodiment using the thresholds.

Controller 101 determines in step S35 whether or not the battery voltage is equal to or greater than threshold Vpremin, and in a case where the battery voltage is less than threshold Vpremin, controller proceeds to step S36 and stops the energy charging.

In a case where the battery voltage is equal to or greater than threshold Vpremin, controller 101 determines in step S37 whether or not the battery voltage is equal to or greater than threshold VpreL, and in a case where the battery voltage is less than threshold VpreL, controller 101 performs additional charging at a low rate in step S38 and outputs a remaining battery level alarm (that is, informs the user by a display and/or sound that the remaining battery level is decreasing).

In a case where the battery voltage is equal to or greater than threshold VpreL, controller 101 determines in step S39 whether or not the battery voltage is equal to or greater than threshold VpreM, and in a case where the battery voltage is less than threshold VpreM, controller 101 proceeds to step S40 and performs the additional charging at a low rate.

In a case where the battery voltage is equal to or greater than threshold VpreM, controller 101 determines in step S41 whether or not the battery voltage is equal to or greater than threshold VpreH, and in a case where the battery voltage is less than threshold VpreH, controller 101 proceeds to step S42 and performs the additional charging at a medium rate. In a case where the battery voltage is equal to or greater than threshold VpreHI, on the other hand, controller 101 proceeds to step S43 and performs the additional charging at a high rate.

Here, the low-rate charging can be realized by decreasing the current value of current controller 114, the middle-rate charging can be realized by setting the current value of current controller 114 to be moderate, and the high-rate charging can be realized by increasing the current value of current controller 114. That is, controller 101 controls, based on the charging rate during the additional charging, the current value output from battery 111 during the additional charging.

In the case of the embodiment, the current that flows into current controller 114 is 1.0 [A] in the low-rate charging, 1.5 [A] in the medium-rate charging, and 2.0 [A] in the high-rate charging. Further, in the case of the embodiment, the time required for storing a desired amount of defibrillation energy in capacitor 115 is 20 seconds in the low-rate charging, 15 seconds in the middle-rate charging, and 10 seconds in the high-rate charging. Note that, in the embodiment, the charging rate is classified into three ranks of the low rate, the medium rate, and the high rate, but as a matter of course, the charging rate is not limited to the three ranks, may be two ranks, and may be classified into four or more ranks.

As described above, defibrillator 100 in the present embodiment performs the charging of capacitor 115 by battery 111 by diving the charging of capacitor 115 by battery 111 into at least the pre-charging and the additional charging subsequent to the pre-charging, further measures the output voltage of the battery during the pre-charging, and controls, based on the measured output voltage, the charging rate during the additional charging.

Thus, it is possible to realize defibrillator 100 and the defibrillation energy charging method each capable of appropriately charging capacitor 115 in accordance with a remaining level of battery 111.

That is, according to the present embodiment, it is possible to measure the remaining level of battery 111 accurately, and further, the additional charging is performed at a charging rate according to the remaining battery level. Therefore, it is possible to perform efficient additional charging while keeping a voltage drop of the battery during the additional charging within a permissible range. As a result, even in a case where the remaining level of battery 111 becomes low, it is possible to avoid the situation where the operations of entire defibrillator 100 stop and to prevent the charging of capacitor 115 with defibrillation energy from becoming unnecessarily long.

According to the configuration of the embodiment described above, the pre-charging and the remaining battery capacity estimation are performed at once, and thus, it is not necessary to add a circuit for applying a load to battery 111 separately for the remaining battery level estimation.

Note that, when capacitor 115 is charged at a constant charging rate (charging current) regardless of the remaining level of battery 111, a situation in which “time is taken for the charging despite that the remaining level of battery 111 is sufficient”, or a situation in which “it is determined as impossible to charge energy despite that there is a remaining level enough to charge energy when the current value is decreased (that is, when the charging rate is decreased)” may occur. According to the configuration of the present embodiment, it is possible to avoid such a situation and to charge appropriate defibrillation energy.

The embodiment described above is only illustration of an exemplary embodiment for implementing the present invention, and the technical scope of the present invention shall not to be construed limitedly thereby. That is, the present invention can be implemented in various forms without departing from the gist or the main features thereof.

In the above-described embodiment, an example in which voltage analyzer 117 is provided separately from controller 101 has been described, but voltage analyzer 117 may be incorporated in controller 101. For example, in a case where controller 101 is a CPU, the voltage may be measured by using a function of CPU.

### REFERENCE SIGNS LIST

100 Defibrillator
101 Controller
111 Battery
114 Current controller
115 Capacitor
116 Power switch
117 Voltage analyzer
200 Discharger (pad or paddle)

1. defibrillator, comprising:
a battery;
a capacitor that stores defibrillation energy;
a controller that is configured to control charging of the capacitor by the battery; and
a voltage analyzer that measures an output voltage of the battery, wherein:
the controller is configured to control the charging of the capacitor by the battery such that the charging of the capacitor by the battery is divided into at least pre-charging and additional charging subsequent to the pre-charging, and
the controller is further configured to control, based on the output voltage of the battery measured by the voltage analyzer during the pre-charging, a charging rate during the additional charging.

2. The defibrillator according to claim 1, wherein
the controller is configured to decrease the charging rate during the additional charging as the output voltage of the battery measured by the voltage analyzer during the pre-charging is smaller.

3. The defibrillator according to claim 1, wherein
the controller is configured to control, based on the charging rate during the additional charging, a current value output from the battery during the additional charging time.

4. The defibrillator according to claim 1, wherein
a current value that flows from the battery to the capacitor during the pre-charging is equal to or less than a current value that flows from the battery to the capacitor during the additional charging.

5. The defibrillator according to claim 1, wherein
the battery is a primary battery.

6. A defibrillation energy charging method in a defibrillator, the method comprising:
pre-charging a capacitor by a battery;
additionally charging the capacitor by the battery after the pre-charging;
measuring an output voltage of the battery during the pre-charging; and
controlling, based on the measured output voltage, a charging rate during the additionally charging of the capacitor.
